(19) United States
(12) Patent Application Publication
(10) Pub. No.: US 2018/0184973 A1
(43) Pub. Date: Jul. 5, 2018

(54) MAGNETIC MEASUREMENT OF FAT FRACTION

(71) Applicant: Krishna Nayak, Long Beach, CA (US)

(72) Inventor: Krishna Nayak, Long Beach, CA (US)

(21) Appl. No.: 15/857,610

(22) Filed: Dec. 28, 2017

Related U.S. Application Data
(60) Provisional application No. 62/440,294, filed on Dec. 29, 2016.

(51) Int. Cl. A61B 5/00; A61B 5/055

(52) U.S. Cl. CPC A61B 5/4872; A61B 5/055

(57) ABSTRACT

The subject technology provides an apparatus for measuring fat content. The apparatus includes a probe, the probe including a magnet array and a radio frequency (RF) subsystem.

### CROSS-REFERENCE TO RELATED APPLICATIONS

The present application claims the benefit of U.S. Provisional Patent Application Ser. No. 62/440,294, entitled “MAGNETIC MEASUREMENT OF FAT FRACTION,” filed Dec. 29, 2016, which is hereby incorporated herein by reference in its entirety and made part of the present U.S. Utility patent application for all purposes.

### TECHNICAL FIELD

The present description generally relates to a device for magnetically testing a section of body tissue (e.g., liver) for a fat content.

### BACKGROUND

Non-alcoholic fatty liver disease (NAFLD) is one of the scenarios of fatty liver, occurring when fat is deposited (steatosis) in the liver due to causes other than excessive alcohol use. NAFLD is the most common liver disorder in developed countries.

### BRIEF DESCRIPTION OF THE DRAWINGS

Certain features of the subject technology are set forth in the appended claims. However, for purpose of explanation, several embodiments of the subject technology are set forth in the following figures.

FIG. 1 provides an example of an apparatus according to at least one embodiment of the subject technology.

FIG. 2 provides an example of an apparatus according to at least one embodiment of the subject technology.

FIG. 3 conceptually illustrates an example showing components of the console and the RF subsystem of the apparatus discussed in FIG. 2 with respect to a measurement environment according to at least one embodiment of the subject technology.

FIG. 4 illustrates an electronic system with which one or more embodiments of the subject technology may be implemented.

### DETAILED DESCRIPTION

The detailed description set forth below is intended as a description of various configurations of the subject technology and is not intended to represent the only configurations in which the subject technology can be practiced. The appended drawings are incorporated herein and constitute a part of the detailed description. The detailed description includes specific details for the purpose of providing a thorough understanding of the subject technology. However, the subject technology is not limited to the specific details set forth herein and can be practiced using one or more other implementations. In one or more implementations, structures and components are shown in block diagram form in order to avoid obscuring the concepts of the subject technology.

Embodiments of the subject technology provide a device that can provide fast, accurate, point-of-care assessment of an organ (e.g., a liver). As a result, non-alcoholic fatty-liver disease status can be monitored more closely because a test enabled by the subject technology may be inexpensive, non-invasive, and safe. In an example, the aforementioned test can be performed in the office of a hepatologist or gastroenterologist. More specifically, embodiments of the subject technology generally describe a nuclear magnetic resonance (NMR) based instrument that can be placed on the surface of a patient's abdomen and can measure liver fat fraction within a large deep voxel (e.g., a volume).

Liver fat has been identified by some as the “best” discriminator between metabolically normal and metabolically abnormal obesity. Although magnetic resonance imaging (MRI) is one way to make this measurement, it may be too expensive, logistically difficult, and not cost-effective for a practical implementation.

Roughly one third of the US population has non-alcoholic fatty-liver disease (NAFLD) although most remain undiagnosed. The current standard for diagnosis is liver biopsy, which is difficult to justify because it is expensive and invasive, leaving an unmet need for an inexpensive and non-invasive device that can quantify liver-fat as a screening tool for NAFLD, for treatment and/or longitudinal monitoring, and for use in diagnostic and prognostic decisions. NAFLD prevalence is on the rise, due in part to the increased prevalence of obesity and metabolic syndrome, and it is linked to increased risk for cardiovascular disease, diabetes, and liver cancer.

Embodiments described herein screen for Metabolic Syndrome (MetS), Nonalcoholic Steatohepatitis (NASH), and NAFLD using a small, mobile diagnostic device or apparatus as described herein. The device can perform a function similar to a MRI system and magnetically test a section of liver for fat quantity.

At least one embodiment provides a system with an estimated cost from $20-50k, about 60-150 times cheaper than an existing MRI machine. The device requires little maintenance and therefore provides a fast, inexpensive, non-invasive test that does not currently exist. Thus, embodiments described herein significantly reduce the barriers for physicians who treat at-risk patients.

The incidence of MetS alone in the US population over 60 years of age is estimated to be nearly 60%. Further, NASH and NAFLD are becoming more prevalent. For example, NASH affects about 2-5% of the US population and there are nearly 3 million cases of NAFLD diagnosed annually. In spite of this need, no existing devices leverage NMR or MRI technology without expensive imaging techniques to non-invasively diagnose liver fat, a proxy for MetS, NASH, NAFLD, and other types of hepatic steatosis (liver fat infiltration).

In view of the above, embodiments of the subject technology may be utilized 1) to noninvasively diagnose liver fat content as a biomarker for MetS, NASH, NAFLD, and other types of hepatic steatosis; and 2) to diagnose intramuscular fat as a biomarker for degenerative muscular disorders such as Duchenne Muscular Dystrophy.

In at least one embodiment, a small magnetic device light enough to be mounted on the wall, a patient table, or a small cart will be designed with a known magnetic field strength and shape. The magnet will achieve tissue polarization at least 5 cm from the magnet surface, the depth of pure liver (excluding overlap with bone and subcutaneous fat) in an estimated 95% of patients. A small, integrated antenna will excite liver tissue and receive signals. The signals will be produced using NMR methods and recorded for analysis.

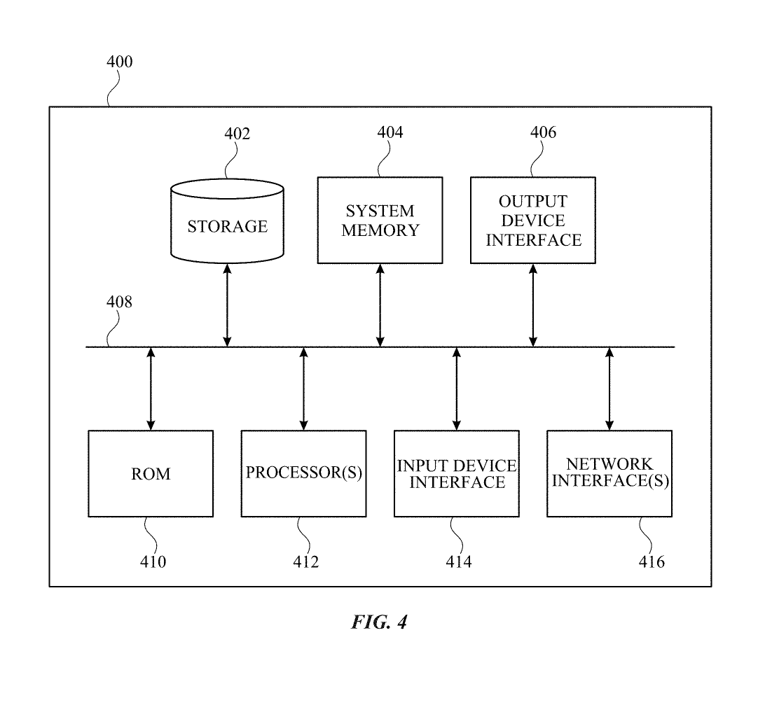

In at least one embodiment, on the device, patterns of electromagnetic energy called pulse sequences will be generated in software executed on a computing device (e.g., as provided by an electronic system in FIG. 4 described further below) and transmitted at different times. Curve fitting techniques may be used to determine the fat content and other tissue properties from the decay rate of the signal. The system will then report these values and indicate whether the patient is healthy or needs a complete examination for liver fat infiltration and the root causes.

In at least one embodiment, the device can leverage NMR technology to diagnose liver fat content. Currently, only invasive, unreliable tests and/or very expensive MRI imaging series are utilized to diagnose the aforementioned conditions. The mobility of small NMR units, as provided by embodiments herein, can provide in-office diagnostics in a way that may have not yet been attempted.

Additionally, embodiments of the subject technology can include a permanent, rare-earth magnet capable of achieving tissue polarization at 3-7 cm to excite liver tissue. Further, such a magnet can have predictable roll-off characteristics. The depth and field shape of such a magnet are novel compared to other existing permanent magnet designs, which are typically designed for uniformity or cost reduction.

At least one embodiment provides an apparatus similar to NMR well logging probes with a permanent magnet at the center and a radio frequency (RF) transceiver module on one side. Although at least one embodiment uses a RF transceiver module, it is appreciated that other embodiments may include a separate RF transmitter module and a separate RF receiver module. Short band-limited RF pulses may be utilized. The iso-field lines of the magnet may limit along an axis into the body. The coil sensitivity could limit signal along the orthogonal axes. Selecting out a single not-too-deep large voxel therefore may be provided. Further, array receivers could be used along with MEG-type (magnetoencephalography type) reconstruction techniques.

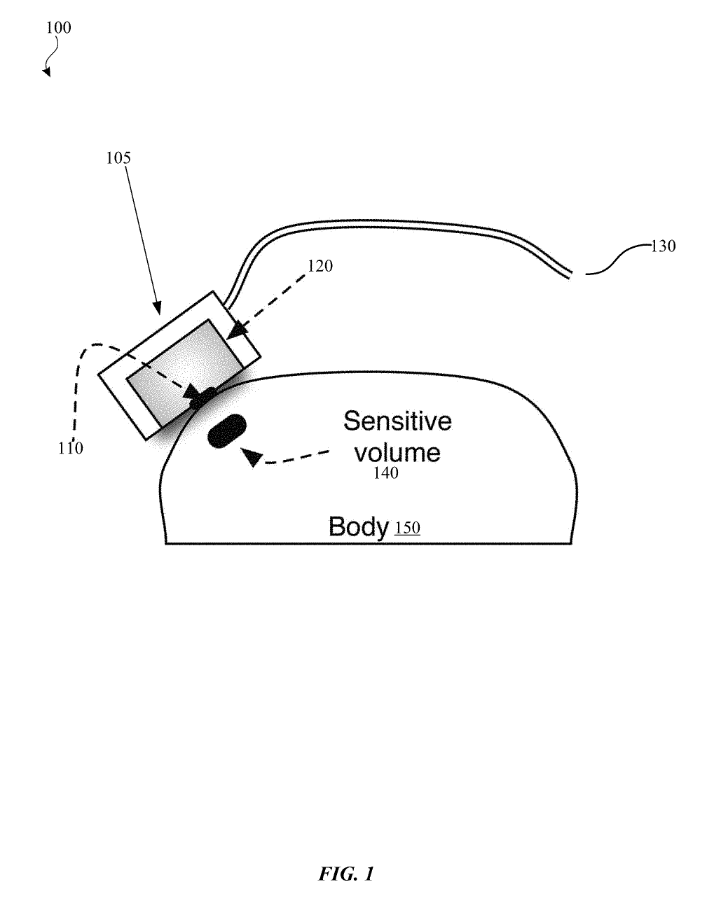

As illustrated in FIG. 1, an apparatus 100 is shown that includes a small magnetic device 105 (e.g., a hand portable probe) for providing a known magnetic field strength and shape in accordance to some embodiments of the subject technology. In an embodiment, the magnetic device 105 includes a magnet array 120 that can achieve tissue polarization at least 5 cm from the surface of the magnet array 120, the depth of pure liver (excluding overlap with bone and subcutaneous fat) in an estimated 95% of patients. One embodiment of the magnet array 120 is a linear Halbach array (e.g. dipole). Another embodiment is a curved linear Halbach array (e.g. dipole) to provide a closer fit to the human torso (or target body region). A small, integrated antenna for radio frequency (RF) signal input/output 130 can excite liver tissue and receive signals. In an example, the integrated antenna may be and/or a part of an RF coil 110. In an example, signals for testing body tissue may be produced using methods similar to MR imaging and recorded for analysis.

In some embodiments, the magnet array 120 can provide tissue polarization at a range from 3-7 cm to excite liver tissue corresponding to a sensitive volume 140 within a body 150. Further, the magnet array 120 can have predictable roll-off characteristics. Utilizing the components described above, the apparatus 100 is configured to measure liver fat fraction where the liver fat fraction includes proton density fat fraction (PDFF). The device 105 in at least one embodiment is a hand-portable probe and/or may be configured to be mounted on a wall, patient table, or a small cart.

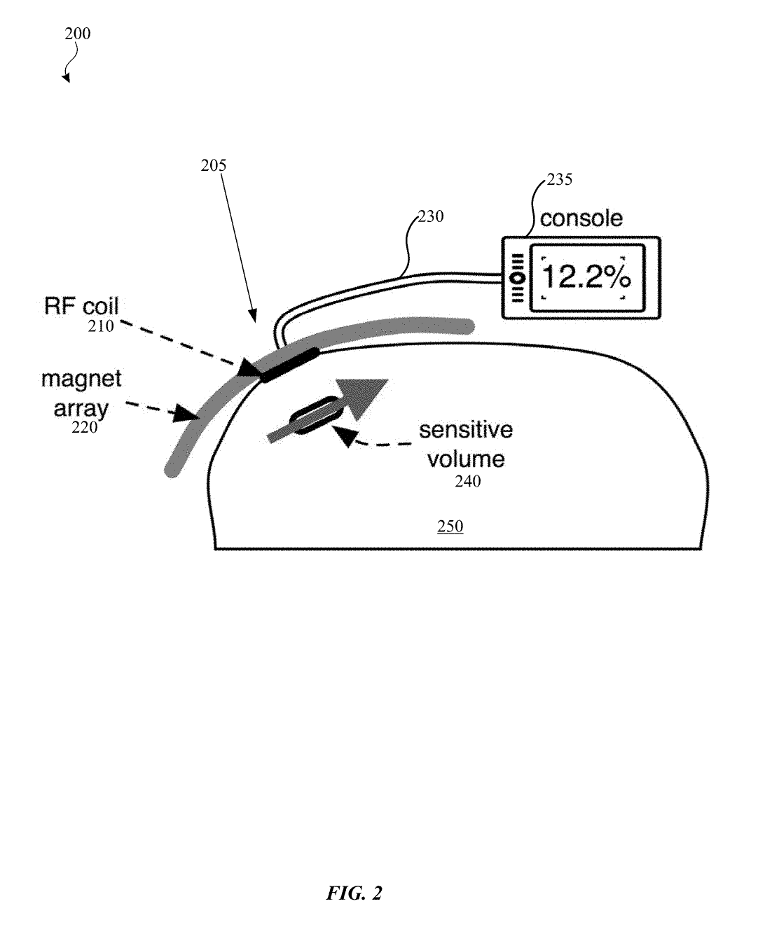

FIG. 2 provides an example of an apparatus 200 according to at least one embodiment of the subject technology.

As illustrated in FIG. 2, an apparatus 200 is shown that includes a small magnetic device 205 (e.g., a hand portable probe) for providing a known magnetic field strength and shape. In an embodiment, the magnetic device 205 includes a magnet array 220 that can achieve tissue polarization at least 5 cm from the surface of the magnet array 220, the depth of pure liver (excluding overlap with bone and subcutaneous fat) in an estimated 95% of patients. One embodiment of the magnet array 120 is a linear Halbach array (e.g., dipole). Another embodiment is a curved linear Halbach array (e.g., dipole) to provide a closer fit to the human torso (or target body region). Thus, in at least one implementation, the magnet array 220 may include a linear or curved linear strip of magnets in a dipole Halbach array pattern. In an example, the strip of magnets may be permanent magnets. A small, integrated antenna for radio frequency (RF) signal input/output can excite liver tissue (e.g., corresponding to a sensitive volume 240) and receive signals. In an example, an RF subsystem 210 may provide the functionality of the antenna for receiving and/or transmitting RF signals. In an example, signals for testing body tissue may be produced using NMR methods and recorded for analysis. The device 205 in at least one embodiment is a hand-portable probe and/or may be configured to be mounted on a wall, patient table, or a small cart.

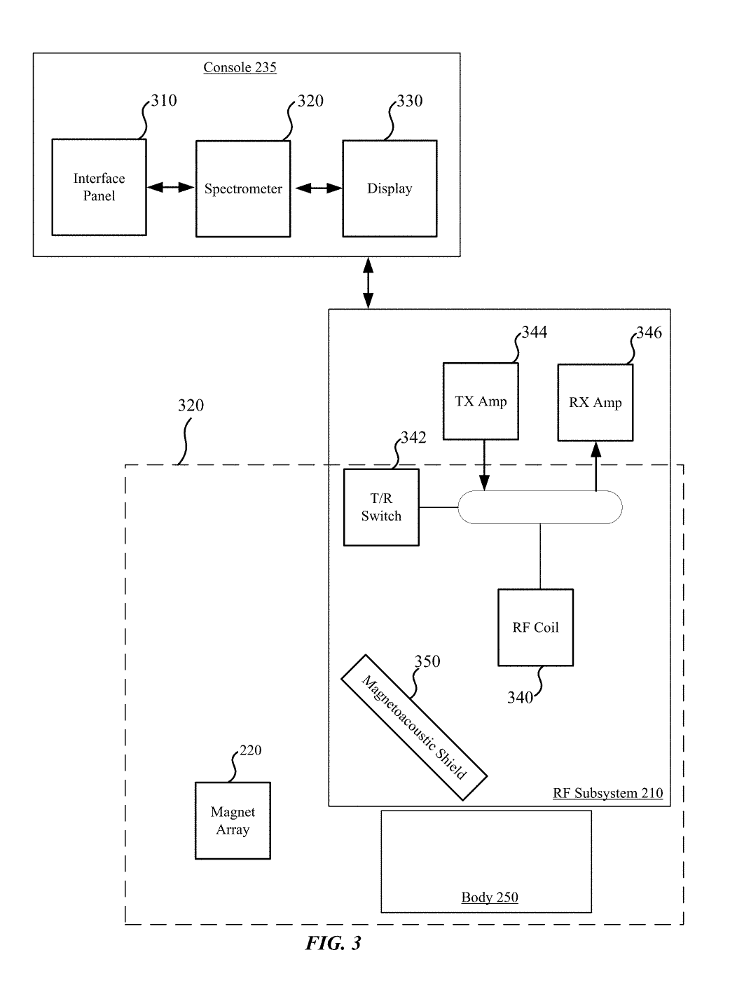

In one or more embodiments, the apparatus 200 includes a console 235. The console 235 provides the functionality of at least a spectrometer, and can include components such as a display, interface panel, spectrometer, RF transmit amplifier, and/or power supply. The console 235 is described further in FIG. 3 below. The RF subsystem 210 may include components such as a transmit/receive RF coil (or coil array), a RF transmit/receive switch, RF low-noise receive amplifier, and/or a magnetoacoustic shield (e.g., thin layer between the RF subsystem 210 and the magnet array 220). As illustrated, the RF subsystem 210 is between the magnet array 220 and a body 250 of a patient. The RF subsystem 210 is described further in FIG. 3 below.

In at least an embodiment, the console 235 is coupled to the RF subsystem 210 through a two-port cable 230 (e.g., co-axial or similar). Although for purposes of explanation, a single RF coil is illustrated, it is appreciated that multiple RF coils may be provided by the apparatus 200. In this embodiment, each RF coil from the multiple RF coils can be coupled to the console 235 via a respective two-port cable.

Utilizing the components described above, the apparatus 200 is configured to measure liver fat fraction where the liver fat fraction includes proton density fat fraction (PDFF).

In at least an embodiment, the magnet array 220 is always on, and does not need to be turned on or off or controlled in any other way. In some embodiments, the magnet array 220 can provide tissue polarization at a range from 3-7 cm to excite liver tissue corresponding to the sensitive volume 240 within the body 250. Further, the magnet array 120 can have predictable roll-off characteristics.

FIG. 3 conceptually illustrates an example showing components of the console 235 and the RF subsystem 210 of the apparatus 200 discussed in FIG. 2 with respect to a measurement environment 320 according to at least one embodiment of the subject technology. FIG. 3 therefore will be discussed by reference to portions of FIG. 2.

As illustrated, the console 235 includes components such as a display 330, an interface panel 310, and spectrometer 320. Although not illustrated, the console 235 may also include a RF transmit amplifier, and/or a power supply. The RF subsystem 210 may include components such as a transmit/receive RF coil (or coil array) 340, a RF transmit/receive switch 342, RF low-noise receive amplifier 346, a RF transmit amplifier 344, and/or a magnetoacoustic shield 350 (e.g., thin layer between the RF subsystem 210 and the magnet array 220). As illustrated, the measurement environment 320, provided by the magnet array 220 and RF subsystem, may include the body 250 in which measurements are to be taken.

Although not illustrated, embodiments described in the apparatus 100 and/or 200 may be stored in a shield that minimizes the magnetic field and interference with other equipment. For example, the shield may include an outer casing that can be placed around the device 105 and/or 205 to minimize the stray field of the magnet array. The shield may be completely passive and not wired to anything, and may be placed on the device 105 and/or 205 when the device 105 and/or 205 is not in use (e.g., when storing in the closet or storage area of a medical practitioner's office).

FIG. 4 illustrates an electronic system 400 with which one or more embodiments of the subject technology may be implemented. In at least an embodiment, the console 235 may be implemented to include all or some of the components of the electronic system 400. The electronic system 400 may include various types of computer readable media and interfaces for various other types of computer readable media. The electronic system 400 includes a bus 408, one or more processing unit(s) 412, a system memory 404 (and/or buffer), a ROM 410, a permanent storage device 402, an input device interface 414, an output device interface 406, and one or more network interfaces 416, or subsets and variations thereof.

The bus 408 collectively represents all system, peripheral, and chipset buses that communicatively connect the numerous internal devices of the electronic system 400. In one or more embodiments, the bus 408 communicatively connects the one or more processing unit(s) 412 with the ROM 410, the system memory 404, and the permanent storage device 402. From these various memory units, the one or more processing unit(s) 412 retrieves instructions to execute and data to process in order to execute the processes of the subject disclosure. The one or more processing unit(s) 412 can be a single processor or a multi-core processor in different embodiments.

The ROM 410 stores static data and instructions that are needed by the one or more processing unit(s) 412 and other modules of the electronic system 400. The permanent storage device 402, on the other hand, may be a read-and-write memory device. The permanent storage device 402 may be a non-volatile memory unit that stores instructions and data even when the electronic system 400 is off. In one or more embodiments, a mass-storage device (such as a magnetic or optical disk and its corresponding disk drive) may be used as the permanent storage device 402.

In one or more embodiments, a removable storage device (such as a floppy disk, flash drive, and its corresponding disk drive) may be used as the permanent storage device 402. Like the permanent storage device 402, the system memory 404 may be a read-and-write memory device. However, unlike the permanent storage device 402, the system memory 404 may be a volatile read-and-write memory, such as random access memory. The system memory 404 may store any of the instructions and data that one or more processing unit(s) 412 may need at runtime. In one or more embodiments, the processes of the subject disclosure are stored in the system memory 404, the permanent storage device 402, and/or the ROM 410. From these various memory units, the one or more processing unit(s) 412 retrieves instructions to execute and data to process in order to execute the processes of one or more embodiments.

The bus 408 also connects to the input and output device interfaces 414 and 406. The input device interface 414 enables a user to communicate information and select commands to the electronic system 400. Input devices that may be used with the input device interface 414 may include, for example, alphanumeric keyboards and pointing devices (also called “cursor control devices”). The output device interface 406 may enable, for example, the display of images generated by electronic system 400. Output devices that may be used with the output device interface 406 may include, for example, printers and display devices, such as a liquid crystal display (LCD), a light emitting diode (LED) display, an organic light emitting diode (OLED) display, a flexible display, a flat panel display, a solid state display, a projector, or any other device for outputting information. One or more embodiments may include devices that function as both input and output devices, such as a touchscreen. In these embodiments, feedback provided to the user can be any form of sensory feedback, such as visual feedback, auditory feedback, or tactile feedback; and input from the user can be received in any form, including acoustic, speech, or tactile input.

Finally, as shown in FIG. 4, the bus 408 also couples the electronic system 400 to one or more networks and/or to one or more network nodes through the one or more network interface(s) 416. In this manner, the electronic system 400 can be a part of a network of computers (such as a LAN, a wide area network (“WAN”), or an Intranet, or a network of networks, such as the Internet. Any or all components of the electronic system 400 can be used in conjunction with the subject disclosure.

Those of skill in the art would appreciate that the various illustrative blocks, modules, elements, components, methods, and algorithms described herein may be implemented as electronic hardware, computer software, or combinations of both. To illustrate this interchangeability of hardware and software, various illustrative blocks, modules, elements, components, methods, and algorithms have been described above generally in terms of their functionality. Whether such functionality is implemented as hardware or software depends upon the particular application and design constraints imposed on the overall system. Skilled artisans may implement the described functionality in varying ways for each particular application. Various components and blocks may be arranged differently (e.g., arranged in a different order, or partitioned in a different way) all without departing from the scope of the subject technology.

It is understood that any specific order or hierarchy of blocks in the processes disclosed is an illustration of example approaches. Based upon design preferences, it is understood that the specific order or hierarchy of blocks in the processes may be rearranged, or that all illustrated blocks be performed. Any of the blocks may be performed simultaneously. In one or more implementations, multitasking and parallel processing may be advantageous. Moreover, the separation of various system components in the implementations described above should not be understood as requiring such separation in all implementations, and it should be understood that the described program components and systems can generally be integrated together in a single software product or packaged into multiple software products.

As used in this specification and any claims of this application, the terms “base station”, “receiver”, “computer”, “server”, “processor”, and “memory” all refer to electronic or other technological devices. These terms exclude people or groups of people. For the purposes of the specification, the terms “display” or “displaying” means displaying on an electronic device.

As used herein, the phrase “at least one of” preceding a series of items, with the term “and” or “or” to separate any of the items, modifies the list as a whole, rather than each member of the list (i.e., each item). The phrase “at least one of” does not require selection of at least one of each item listed; rather, the phrase allows a meaning that includes at least one of any one of the items, and/or at least one of any combination of the items, and/or at least one of each of the items. By way of example, the phrases “at least one of A, B, and C” or “at least one of A, B, or C” each refer to only A, only B, or only C; any combination of A, B, and C; and/or at least one of each of A, B, and C.

The predicate words “configured to”, “operable to”, and “programmed to” do not imply any particular tangible or intangible modification of a subject, but, rather, are intended to be used interchangeably. In one or more implementations, a processor configured to monitor and control an operation or a component may also mean the processor being programmed to monitor and control the operation or the processor being operable to monitor and control the operation. Likewise, a processor configured to execute code can be construed as a processor programmed to execute code or operable to execute code.

Phrases such as an aspect, the aspect, another aspect, some aspects, one or more aspects, an implementation, the implementation, another implementation, some implementations, one or more implementations, an embodiment, the embodiment, another embodiment, some implementations, one or more implementations, a configuration, the configuration, another configuration, some configurations, one or more configurations, the subject technology, the disclosure, the present disclosure, other variations thereof and alike are for convenience and do not imply that a disclosure relating to such phrase(s) is essential to the subject technology or that such disclosure applies to all configurations of the subject technology. A disclosure relating to such phrase(s) may apply to all configurations, or one or more configurations. A disclosure relating to such phrase(s) may provide one or more examples. A phrase such as an aspect or some aspects may refer to one or more aspects and vice versa, and this applies similarly to other foregoing phrases.

The word “exemplary” is used herein to mean “serving as an example, instance, or illustration”. Any embodiment described herein as “exemplary” or as an “example” is not necessarily to be construed as preferred or advantageous over other implementations. Furthermore, to the extent that the term “include”, “have”, or the like is used in the description or the claims, such term is intended to be inclusive in a manner similar to the term “comprise” as “comprise” is interpreted when employed as a transitional word in a claim.

All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. § 112, sixth paragraph, unless the element is expressly recited using the phrase “means for” or, in the case of a method claim, the element is recited using the phrase “step for”.

The previous description is provided to enable any person skilled in the art to practice the various aspects described herein. Various modifications to these aspects will be readily apparent to those skilled in the art, and the generic principles defined herein may be applied to other aspects. Thus, the claims are not intended to be limited to the aspects shown herein, but are to be accorded the full scope consistent with the language claims, wherein reference to an element in the singular is not intended to mean “one and only one” unless specifically so stated, but rather “one or more”. Unless specifically stated otherwise, the term “some” refers to one or more. Pronouns in the masculine (e.g., his) include the feminine and neuter gender (e.g., her and its) and vice versa. Headings and subheadings, if any, are used for convenience only and do not limit the subject disclosure.

What is claimed is:

1. An apparatus for measuring fat content comprising:
a probe, the probe including a magnet array and a radio frequency (RF) subsystem.

2. The apparatus of claim 1, wherein the apparatus is configured to measure liver fat fraction, the liver fat fraction including proton density fat fraction (PDFF).

3. The apparatus of claim 1, where the probe comprises a hand-portable probe or configured to be mounted on a wall, patient table, or a small cart.

4. The apparatus of claim 1, further comprising:
a radio frequency (RF) transceiver configured to excite liver tissue and receive RF signals, the RF transceiver coupled to at least one antenna for receiving the RF signals.

5. The apparatus of claim 1, wherein the probe is configured to achieve tissue polarization at least 3 centimeters from a surface of the magnet array.

6. The apparatus of claim 1, wherein the magnet array further comprises:
a linear or curved linear strip of magnets in a dipole Halbach array pattern.

7. The apparatus of claim 6, wherein the strip of magnets further comprises permanent magnets.

8. The apparatus of claim 1, wherein the RF subsystem further comprises:
a transmit/receive RF coil, RF transmit/receive switch, RF receive low-noise amplifier, or a magnetoacoustic shield.

9. The apparatus of claim 1, further comprising:
a console connected to the RF subsystem through at least one cable.

10. The apparatus of claim 9, wherein the console further comprises:
an interface panel, spectrometer, display, or power supply.
